(19) United States
(12) Patent Application Publication
(10) Pub. No.: US 2006/0064771 A1
(43) Pub. Date: Mar. 23, 2006

(54) Procedure of genetic recombination for galinaceae hybrids breeding

(76) Inventor: Florin Pricop, Bucharest (RO)

Correspondence Address: HERSHKOVITZ & ASSOCIATES, 1725 I STREET NW, SUITE 300, WASHINGTON, DC, 20006, US

(21) Appl. No.: 10/539,259

(22) PCT Filed: Sep. 16, 2003
(86) PCT No.: PCT/RO03/00013

§ 371 Date: Jun. 16, 2005

(30) Foreign Application Priority Data
Aug. 8, 2003 (RO) 2003 00678

(51) Int. Cl. A01K 67/027

(52) U.S. Cl. 800/19; 119/300

(57) ABSTRACT

The invention refers to a procedure of genetic recombination for Galinaceae hybrids breeding, specialized in the production of eggs for consumption. For the achievement of genetic recombination, parents originating from pure homozygous lines for barred (B) and gold (b) heterosomal lines, playing a role in the transmission of feather colour to the resulting hybrid chicks, were selected. By crossing a homozygous recessive (bb) red Rhode Island male with homozygous dominant (BB) Marans female, two categories of phenotypes, one for each sex, of equal proportion, resulted in F1 generation. The feather colour is genetically determined by the activity of gold and barred genes, present both in the genotype of the heterozygous (Bb) males and in the genotype of the heterozygous (bB) females, the sex being genetically determined by the dominant sex gene, SDW, located in chromosome W and by the recessive sex gene, sdw, located in chromosome Z. The heterozygous genotype SDWsdw determines the female sex, while the recessive homozygous genotype sdwsdw determines the male sex. The non-allelic interaction of the dominant sex gene on the barred gene overlaps the allelic interaction between the barred and gold genes, the latter one becoming non-functional, and as a consequence the heterozygous females show a different genotype from those observed in the heterozygous males. This feature allows day-old chicks sex screening according to the colour of the juvenile feathers. The results obtained show that the two monitored characters, the colour of feathers and the sex, are genetically determined by the action of the particular genes and that between the genes coding the two characters there is a linkage both in chromosome Z and in chromosome W.

The invention refers to a procedure of genetic recombination for Galinaceae hybrids breeding, specialized in the production of eggs for consumption.

The classical system of selection is known for the breeding of the existing breeds and lines of layers, consisting in the reproductive isolation of the valuable gene funds and their wide use for reproduction, concomitantly with the removal of the undesired genotypes.

However, one limit of the classical system of breeding is that the use of these breeds and lines in the industrial system resulted in attaining the peak selection performance (the genetic plateau), namely a production of around 220 eggs per layer.

This is due to the used system of selection, which is based only on the active gene interactions, which increases the frequency of homozygote within the populations, homozygous trait compensated by other epistatic interactions, which maintained them in a genetic balance.

The increase of homozygote frequency beyond the limits of the genetic balance, with the view to improve the laying percentage, caused the depression of consanguinity and the onset of a genetic drift. Therefore, going beyond the selection plateau can not be done only by the classical methods of selection applied on the stock of layers.

Another problem insufficiently investigated, and therefore not yet clarified, concerns the genetic mechanism and the biochemistry of sex determination in poultry. Because sex determination in poultry is a complex process, the identification of sex specific markers is of particular importance. Very useful are particularly those markers that can be used to identify the sex of immature birds, before the specific morphological traits appear (secondary sexual characters such as the comb and the wattles). The early identification of sex in immature birds is of special importance to poultry breeding because allows the removal from reproduction and subsequent growth of the undesired genotypes (hybrid male).

In accordance with the present invention the procedure used to identify and screen sexes before sexual maturation is based on the colour of the juvenile feathers. The use of this phenotypic marker as screening tool, followed by a program of directed crossing, leads to performing genetic recombinant birds as to their morphology and production.

The technical issue of the invention consists in the induction of genetic recombination at the level of F1 generation, followed by progeny screening by sex as the result of sex genes interaction, based on the use of juvenile feathers colour, as phenotypic marker.

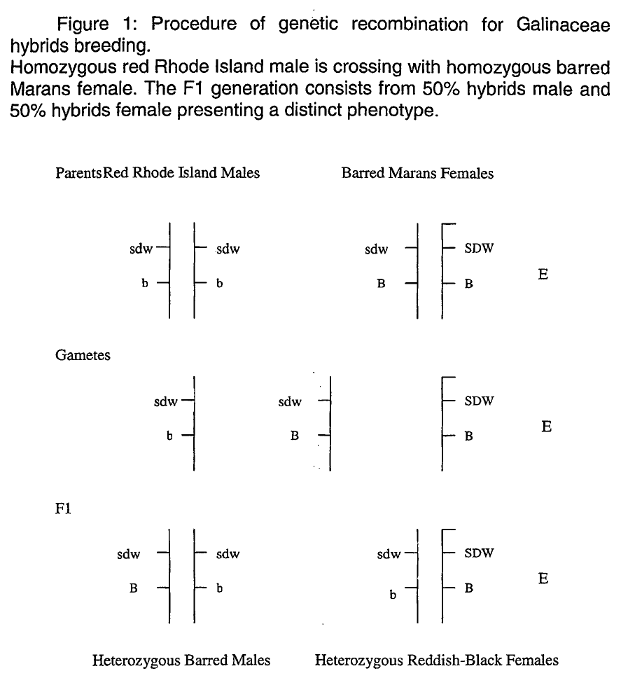

The procedure of genetic recombination for Galinaceae breeding, according to the invention, consists in the fact that, after the selection of parents, originating from pure, homozygous lines for the transmission of feathers colour, a red Rhode Island male is crossed with a barred Marans female, resulting the F1 generation (FIG. 1).

The hybrid F1 progeny is sexed according to the colour of feathers at day-old. When the progeny reaches the sexual maturity the hybrid males and females are crossed, producing the F2 generation, also separated by phenotype categories, according to the colour of feathers.

The progeny resulting from a homozygous recessive (bb) red Rhode Island male crossed with a homozygous dominant (BB) Marans female, genetically assessed when day-old, consists of 50% heterozygous males (Bb), with black juvenile feathers on'the body and a white spot on the head, and 50% heterozygous females (bB) with black juvenile feathers on the body and on the head.

The progeny F2 resulting from a heterozygous male (Bb) crossed with a heterozygous female (bB) both from F1 generation, genetically assessed when day-old, consists of 49.4% mixture of homozygous (BB) and heterozygous (Bb) females and males with black juvenile feathers on the body and a white spot on the head, 25.1% heterozygous (bB) females and males with black juvenile feathers on the body and head and 25.5% recessive homozygous (bb) females and males with red juvenile feathers on the body and head.

The F2 offspring, assessed genetically at 18 weeks old, consists of 24.7% barred homozygous (BB) females and males with barred feathers, 25.1% reddish-black heterozygous (bB) females and males with black feathers on the body and reddish-black feathers on the neck and head, 25.5% homozygous (bb) females and males with red feathers and 24.7% barred heterozygous (Bb) females and males. The phenotype distribution of male in the last group share between 71.8% with barred feathers and 28.2% with barred feathers on the body and red on the neck and head, while the females are 100% barred.

The phenotypic assessment of F1 generation at 18 weeks old shows 50% of the heterozygous (bB) females with black feathers on the body and reddish-black on the neck and head, which is a combination of colours different both from the red feathers of the homozygous (bb) male parent and from the barred feathers of the heterozygous (Bb) hybrid males from F1. This feature is as a result of the dominant sex gene (SDW), located in the chromosome W of the heterozygous (bB) females with an epistatic action which favours day-old recombinants sexing by the colour of their juvenile feathers. In relation with the recessive allele (sdw) located in a homologous area in chromosome Z, determines the formation of the heterozygous genotype (SDWsdw) of female sex, while the recessive sex gene (sdw) in a homozygous state forms the recessive homozygous genotype (sdwsdw) of male sex.

The advantages of the invention are:

highlighting the gene mechanism of feather colour transmission;
using the colour of feather as phenotypic marker for sexing day-old chicks;
using the colour of feather as marker in highlighting the epistatic effect of the dominant sex gene on the barred gene, both located in chromosome W;
increased morphological and productive performance compared to other hybrids for egg production.

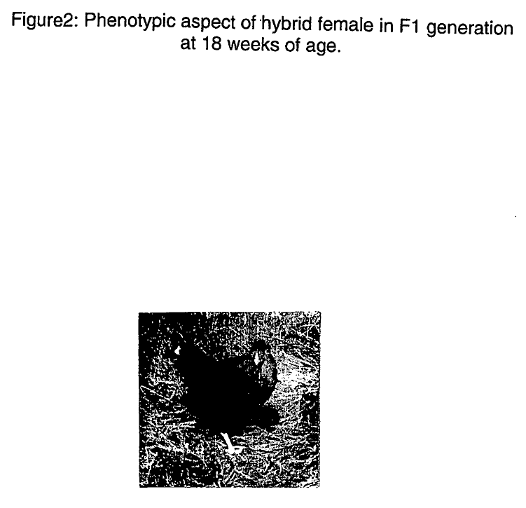

Follow up with an example of procedure, according to the invention, and in connection with the FIG. 1 showing the diagram of producing the genetic recombinants, and with the FIG. 2 showing F1 female phenotype.

A total of 3275 F1 generation day-old chicks hatched, in two batches, from the incubated eggs resulted by crossing a red Rhode Island male with a barred Marans female. The 2633 adult individuals resulted were macroscopic examined for the colour of their feathers at the age of 18 weeks.

By crossing F1 generation offspring (hybrid male and hybrid female) a total of 2440 F2 generation hybrid day-old chicks were produced after two hatching sessions, and 2294 chicks were examined for the colour of their feathers at the age of 18 weeks.

The procedure according to the invention includes the following steps:
Step 1: Selection of the parents originating from pure lines, homozygous for feather colour transmission.

Thus:
a) the male parent, phenotypic with red feathers, while genotypic it is homozygous (bb) for the gold gene (b),
b) the female parent, phenotypic with barred feathers, while genotypic it is homozygous (BB) for the barred gene (B);
Step 2: Crossing the homozygous (bb), red Rhode Island male with homozygous (BB) barred Marans female and production of F1 generation of hybrids displaying of two categories phenotypes, one for each sex (FIG. 1).

Sex screening of day-old hybrids according to the colour of the juvenile feathers is done as follows:
the hybrids males have black juvenile feathers with a white spot of variable size on the head, while genotypic they are heterozygous (Bb);
the hybrids females have black juvenile feathers on the body and head, while genotypic they are heterozygous (bB).

Characteristic to the transmission of the colour of the juvenile feathers to hybrid day-old chicks, is that the male parent (ZZ) transmits the gold gene located on chromosome Z to both sexes in F1 generation.

In the female parent (ZW), the barred gene is located both on chromosome Z and W. The barred gene is transmitted together with chromosome Z only to the hybrid males and through chromosome W only to hybrid females.

The first observations on the hybrid, combination are obtained after day-old chick sexing, when the hybrid males are eliminated because they have no economic importance, while the hybrid females are taken to specially fitted areas for growth and exploitation for production of eggs for consumption.

Monitoring the feather colour of the hybrid male, at the age of 18 weeks, 71.8% of them show barred feathers, while 28.2% have barred feathers on the body and red feathers on the neck and head, but all hybrid female has black feathers on the body and reddish-black feathers on the neck and head.

The colour of hybrid female feathers is different from the red colour of the male parents. Thus, obvious differences were observed between the feather colour of the male parent and the hybrid female produced in generation F1, which shows that the hemizygoucy mechanism does not work at least in the case of feather colour transmission. These differences are explained by the presence of the gold gene (b) in chromosome Z and of the barred gene (B) in chromosome W in the hybrid female, which has the heterozygous bB genotype.

The existence of a heterozygous genotype with a role in feather colour transmission in both sexes in the F1 generation, and the two categories of phenotypes determined according to the feather colour, which allow sexing, shows that in the genetic determinism of the colour of hybrid female feathers, a third gene is acting besides the barred (B) and gold (b) genes. The action of the third gene determines the sex screening of the female and male hybrid according to the colour of the juvenile feathers.

Hybrid chicks sexing by the colour of the juvenile feathers is explained by the action of the SDW dominant sex gene, located in chromosome W, on the barred gene.

The SDW gene plays two functions:
It is a dominant sex gene in relation to its recessive allele sdw, located in the homologous region in chromosome Z. The heterozygous genotype SDWsdw determines the female sex, while the recessive homozygous genotype sdwsdw determines the male sex;
The second, it is functioning as epistatic gene which interacts with the barred gene also located in chromosome W. The non-allelic interaction (E) of the dominant sex gene (SDW): on the barred gene (B), overlaps the allelic interaction between the barred and gold genes, the latter one becoming non-functional and determining in the hybrid female the appearance of only one phenotype category, black feathers on the body and reddish-black feathers on the neck and head.

The colour of the feather observed to the hybrid females is determined by the action of the gold gene located in the chromosome Z and the hypostatic-barred gene located in the chromosome W.

The linked transmission of the genes involved in the feather colour and sex determinism in poultry was shown experimentally by the disagreements between the feathers colour of the male parent and hybrid female. In the crossings presented in the patent and in all cases of crossing of male parent recessive homozygous and the female parent dominant homozygous colour of the feathers of hybrid female in F1 generation is marker for the dominant sex gene.

Step 3: Crossing the F1 offspring between them and production of F2 generation.

Following the crossing of hybrid Bb male with hybrid bB female, three categories of phenotype of day-old chicks are obtained, both sexes equally represented.
49.4% mixture of homozygous (BB) and heterozygous (Bb) females and males with juvenile black feathers on the body and a white spot on the head;
25.1% heterozygous (bB) females and males, with black juvenile feathers on the body and head;
25.5% homozygous (bb) females and males with red juvenile feathers on the body and head.

At 18 weeks of age, the progeny displayed a great variability of the feathers colour, resulting four categories of phenotypes in males and three categories of phenotypes in females, respectively three categories of genotypes, in which both sexes are represented in equal proportions, as follows:
24.7% homozygous (BB), barred females and males with barred feathers;
24.7% heterozygous (Bb) barred females and males, 71.8% of the males having barred feathers and 28.2% of them having barred feathers on the body and red feathers on the neck and head, while all females presented barred feathers;
25.1% heterozygous (bB), reddish-black females and males with black feathers on the body and reddish-black feathers on the neck and head;
25.5% homozygous (bb), females and males with red feathers;
   Unlike the males, which display four categories of phenotypes, the females only have three categories of phenotypes because the mixture of homozygous (BB) and heterozygous (Bb). Barred females have fully barred feathers on the body, neck and head and the assignment of these females in the two phenotypic categories described earlier was done arbitrarily.

The cause-effect relationship, namely the gene-colour relationship, shows the presence of the barred gene in chromosome W in the F1 hybrid female and it is explained by the results of crossing the two heterozygous, namely the Bb male and the bB female, which yielded three categories of genotypes in generation F2, compared to two categories of genotypes obtained by T. H. Morgan (1919).

The presence of the barred gene in the chromosome W was not reported by T. H. Morgan (1919), who crossed Langshan males with barred Plymouth Rock females, which allowed the development of the procedure of producing this hybrid whose colour is also determined by the presence of the barred gene in chromosome W.

In generation F2, the colour of feathers is transmitted in both sexes for each of the categories of phenotypes that resulted, so that sexing the recombinants according to this trait is only possible in generation F1.

The presence of the barred gene in chromosome W in F1 hybrid hens requires the review of the map of heterosomes, modified by F. B. Hutt in 1960. The necessity of reviewing this map is also due to the fact that in the heterosomes there is only one polyallelic locus, for the genes playing a role in the genetic determinism of feathers colour, and not two loci, one for the gold and silver genes and the other for the barred and non-barred genes, as the current map of heterosomes shows.

It was observed that in order to draw the map of heterosomes and to determine only one polyallelic locus in which the genes determining the colour of feathers are located, it must be taken into account that the heterozygous F1 reddish-black females have 100% black feathers on the body and reddish-black feathers on the neck and head, being determined genetically by the gold and barred genes, which form the heterozygous genotype bB. The fact that all females have the same phenotype is due to the linked transmission of the two traits, the colour of feathers and the sex, concomitantly with overlapping the non-allelic interaction of the dominant sex gene (E) on the barred gene with the allelic interaction between the barred and gold genes, the latter becoming non-functional.

The existence of the non-allelic and allelic interaction, overlapping genetic phenomena occurring simultaneously, explains the black feathers on the body and the reddish-black feathers on the neck and head observed in the hybrid (bB) female, which differ from the feathers of both the male parent (bb) and of the hybrid male (Bb).

Unlike the hybrid (bB) female, in the hybrid (Bb) male, only the allelic interaction between the barred and gold genes is present. In the hybrid male no epistatic action of the recessive sex gene (sdw) was noticed on the gold and barred genes located in the heterosome pair ZZ. The result of investigations shows that the genes of the linkage group W have a particular manner of action within the poultry genome due to the epistatic action of the dominant sex gene (SDW).

Respecting the actual map of heterosomes, the colour of feathers in the heterozygous reddish-black females is determined by the action of the gold genes from the locus of silver and gold genes and of the barred gene from the locus of barred and non-barred genes. A non-allelic interaction is considered to be between the gold and barred genes. Phenotypic, was observed that not the epistasy, but rather the incomplete dominant leads to the barred feathers on the body and red feathers on the neck and head in 28.2% of the F1 hybrid males.

The investigations show that the allelic interaction between the barred and gold genes was observed in the hybrid males and should have also been noticed in the F1 hybrid females. However, phenotypic, the allelic interaction between the barred and gold genes in the hybrid females is not functional due to the epistatic action of the dominant sex gene on the barred gene. Due to this fact, the hybrid (bB) females differ phenotypic both from the colour of the male parents (bb) and from the colour of the F1 hybrid males (Bb).

In order to obtain further information on the activity of the heterosomal barred and gold genes, parallel crossing between red Rhode Island males and barred Marans females and barred Marans males and red Rhode Island females was done. In F1 generation 72% hybrid males showed barred feathers on the body, neck and head, and 28% barred feathers on the body and red feathers on the neck and head. Unlike the hybrid males, the F1 hybrid females displayed barred feathers on the body, neck and head. However, a low number of hybrid females, lower than 0.1%, displayed a reddish shadow on the neck and head, over the barred design of the feathers. The red feathers on the neck and head in some hybrid females shows the presence of the gold gene in chromosome W and the epistatic action of the dominant sex gene on the gold gene.

By crossing the hybrid (Bb) males with hybrid (Bb) females from generation F1, three categories of genotypes resulted in generation F2, showing both the heterozygous (Bb): phenotype observed in the hybrid females and the presence of the gold gene in chromosome W.

Similar results were observed for the pair of genes barred—gold and silver—gold. Thus, crossing the homozygous (ss) red Rhode Island males with homozygous (SS) white Rhode Island females, in the F1 hybrid (sS) females preponderantly red feathers were noticed, except for some white feathers accounted by the presence of the silver gene in the chromosome W.

In generation F1, 86.7% of the hybrid (Ss) males had white feathers, the balance of 13.3% displaying white feathers with a few red feathers. Crossing hybrid (Ss) males with hybrid (sS) females, both from generation F1, the resulting F2 generation produced three categories of genotypes in which both sexes are equally represented.

In parallel with the crossing of (ss) red Rhode Island males with (SS) white Rhode Island females, homozygous (SS) white Rhode Island males were crossed with (ss) homozygous white Rhode Island females. In F1 generation, the hybrid (Ss) males have similar feather colour with the hybrid (Ss) males which resulted from the previous crossing. The hybrid (Ss) females had white feathers, except for 0.7% of them, which have a few red feathers scattered in the white feathers of the body, this phenotype being determined by the gold gene (s) acting under the epistatic effect of the dominant sex gene, both gene being in this case located in chromosome W.

Another crossing used homozygous (bb) red Rhode Island males, the place of the homozygous (BB) barred Marans female being taken by homozygous (BB) bared Plymouth-Rock females. The results obtained in generation F1 on the colour of feathers support the results obtained by crossing homozygous (bb) red Rhode Island males with homozygous (BB) barred Marans females.

The day-old chicks from generation F1 displayed two phenotypes, one for each sex. The sexing of the day-old hybrid mix according to the colour of feathers showed that:
phenotypic, the hybrids males have black juvenile feathers with a white spot of variable size on the head, while genotypic they are heterozygous (Bb);
phenotypic, the hybrids females have black juvenile feathers on the body and head, while genotypic they are heterozygous (bB).
   After 18 weeks age, 72% of the hybrids males (Bb) had barred feathers, while 28% of them had barred feathers on the body and red feathers on the neck and head. All hybrids (bB) females had black feathers on the body and reddish-black feathers on the neck and head.

Crossing the gold (bb) males with barred (BB) females, the F1 hybrid females displayed different feather colour than the male parent, while crossing the barred (BB) males with gold (bb) females, the F1 hybrid females displayed barred feathers on the body, neck and head, identical with the feathers of the male parent, except up to 0.1%, which display a red shadow on the neck and head. We propose the introduction in the map of heterosomes of the locus of the genes playing a role in the genetic determinism of the sexes, as follows:
the dominant sex gene, SDW, located in chromosome W;
the recessive sex gene, sdw, located in chromosome Z.
   The results obtained with the F1 hybrid females (FIG. 1) show that the dominant sex gene (SDW) is linked with the barred (B) gene, both genes being located in chromosome W, while the recessive sex gene (sdw) is linked with the gold (b) gene, both located in chromosome Z.

The linked transmission of the genes which determine the colour of the feathers and the sex is characterized by the presence of two loci in chromosome Z, with corresponding similar loci located in chromosome W.

Considering the results of the investigations in the genetic determinism of the colour of feathers and of the sex, we propose the “Gene theory of sexuality”, which is a continuation of the “Chromosomal theory of sex determination”.

The procedure of genetic recombination for breeding the Galinaceae hybrids specialised in the production of eggs for consumption, according to the invention, is different from the known hybrids by the fact that the transmission of feathers colour is explained by a new gene mechanism, while the productive performances are improved compared to other laying hybrids.

Considering an increased number of eggs per production cycle, a lower feed intake for one kg egg and an improved viability, the characteristics of the new hybrid will be presented by the FIG. 1 and FIG. 2 showing the F1 hybrid female with the following characteristics: average size, elongated head, simple, vertical, teethed, bright red comb, large, vivid eyes, slightly bent, strong, yellowish-black beak, red wattles, averagely long neck properly covered by hackles the trunk frames within a round rectangle and has an horizontal position, long, horizontal back, wide breast, full, properly rounded, slightly bent towards the front, medium size wings, properly closed, displayed horizontally, strong feet dressed in feathers, black feathers on the body and reddish-black feathers on the neck and head.

The procedure of obtaining the new hybrid according to the invention relies on the cross between the red. Rhode Island males with barred Marans females, as shown in the FIG. 1.

The productive traits of this hybrid are as follows: 321 eggs by fed layer by production cycle until the age of 77 weeks, average egg weight of 60.9 g and 64.9 g at 34 and 70 weeks, 20.1 kg egg mass with a feed conversion ratio of 2.25 kg feedstuff for one kg of egg mass, average female weight of 2130 g at 34 weeks old, 50% laying percentage at 22 weeks, 96% and 95% viability of the young and adult females, respectively. This is a calm hybrid resistant to the diseases.

1-5. (canceled)

6. Procedure of genetic recombination for Galinaceae hybrids breeding based on the linked transmission of the genes coding for the sex and the feathers color, in which the cross of a recessive (bb) homozygous red Rhode Island male with a dominant (BB) homozygous Marans female yielded in F1 generation 50% heterozygous (Bb) males with black juvenile feathers on the body and a white spot on the head and 50% heterozygous (bB) females with black juvenile feathers, wherein, after 18 weeks growing of F1 progeny, have placed crossing the hybrid F1 heterozygous (Bb) males with black juvenile feathers on the body and a white spot on the head with the heterozygous (bB) females with black juvenile feathers on the body and head resulted the F2 generation, which was assessed genetically by the feathers color when day-old and at the age of 18 weeks.

7. Procedure according to claim 6, wherein day-old sexing of the hybrid F2 progeny showed 49.4% mixture of dominant (BB) homozygous and heterozygous (Bb) males and females with black juvenile feathers on the body and a white spot on the head, 25.1% heterozygous (bB) males and females with black juvenile feathers on the body and head, 25.5% homozygous (bb) females and males with red juvenile feathers on the body and head.

8. Procedure according to the claim 6, wherein, at the age of 18 weeks, F2 progeny showed 24.7% dominant (BB) homozygous females and males with barred feathers, 25.1% heterozygous (bB), reddish-black females and males with black feathers on the body and reddish-black feathers on the neck and head, 25.5% recessive homozygous (bb), females and males with red feathers, 24.7% heterozygous (Bb) barred females and males.

9. Procedure according to claim 6, wherein, 24.7% of the heterozygous (Bb) are barred females and males, 71.8% of the males had barred feathers and 28.2% of the males had barred feathers on the body and red feathers on the neck and head, while 100% females showed barred feathers.

10. Procedure according to claim 8, wherein, 24.7% of the heterozygous (Bb) are barred females and males, 71.8% of the males had barred feathers and 28.2% of the males had barred feathers on the body and red feathers on the neck and head, while 100% females showed barred feathers.

11. Procedure according to claim 6, wherein the heterozygous (bB) F1 females have black feathers on the body and reddish-black feathers on the neck and head, which is different both from the red feathers of the homozygous (bb) male parent and from the barred feathers of the heterozygous males (Bb), this is due to the dominant sex gene (SDW) located on the chromosome W with epistatic action on the barred gene, which allows day-old sexing of the recombinant hybrids by the feather color, and which, in relation with the recessive (sdw) allele located on the chromosome Z, determines the formation of the heterozygous (SDWsdw) female genotype, while in relation with the recessive (sdw) sex gene present in both chromosomes Z, forms the recessive homozygous (sdwsdw) male genotype.

12. Procedure according to the claim 8, wherein the heterozygous (bB) F1 females have black feathers on the body and reddish-black feathers on the neck and head, which is different both from the red feathers of the homozygous (bb) male parent and from the barred feathers of the heterozygous males (Bb), this is due to the dominant sex gene (SDW) located on the chromosome W with epistatic action on the barred gene, which allows day-old sexing of the recombinant hybrids by the feather color, and which, in relation with the recessive (sdw) allele located on the chromosome Z, determines the formation of the heterozygous (SDWsdw) female genotype, while in relation with the recessive (sdw) sex gene present in both chromosomes Z, forms the recessive homozygous (sdwsdw) male genotype.
